(19) United States
(12) Patent Application Publication
(10) Pub. No.: US 2010/0233152 A1
(43) Pub. Date: Sep. 16, 2010

(54) Use of HMGB proteins and nucleic acids that code therefor

(76) Inventor: Jorn Bullerdiek, Bremen (DE)

Correspondence Address: Lisa Mueller;c/o Polsinelli Shughart PC, 161 N. Clark Street, Suite 4200, Chicago, IL, 60601, US

(21) Appl. No.: 10/499,754

(22) PCT Filed: Jun. 18, 2004
(86) PCT No.: PCT/EP2002/014579

§ 371 Date: Jun. 22, 2009

(30) Foreign Application Priority Data
Dec. 19, 2001 (DE) 101 62 556.1

(51) Int. Cl. A61K 39/395; C07K 14/00; C07H 21/04; C07K 16/18; A61K 38/16; A61K 31/7088; A61P 15/18

(52) U.S. Cl. 424/130.1; 530/350; 536/23.5; 530/387.1; 514/12; 514/44 R

(57) ABSTRACT

The invention relates to the use of HMGB and/or a nucleic acid that codes therefor and/or an interaction partner of HMGB that is in particular natural and/or a nucleic acid that codes therefor as a target molecule for the development and/or production of a medicament for the treatment and/or prevention of diseases of the endometrium and/or for the development and/or production of a diagnostic agent for diagnosing diseases of the endometrium.

The present invention relates to the use of HMGB, of a nucleic acid that codes therefor, of an interaction partner of HMGB that is in particular natural and/or a nucleic acid that codes therefor as target molecule for the development of a medicament and/or a diagnostic agent, the use of chemical compounds entering into interaction with HMGB, with a nucleic acid that codes therefor, with an interaction partner of HMGB that is in particular natural and/or with a nucleic acid that codes therefor, for the production and/or development of a medicament and/or a diagnostic agent, a pharmaceutical composition and a kit.

The high mobility group proteins are small, chromatin-associated non-histone proteins which, as a result of their functional sequence motives, among other things, are divided into three families: the HMGB family, the HMGN family and the HMGA family (Bustin M., Revised nomenclature for high mobility group (HMG) chromosomal proteins. Trends Biochem. Sci. 2001, 26:152-1533). The allocation to a family is made on the basis of the DNA binding domains. The proteins of the HMGB family possess so-called DNA boxes as binding domains. The letter B in the abbreviation of the family name consequently stands for box. HMGB1 is the protein of the HMGB family which has been investigated most thoroughly to which HMGB2, HMGB3 and SP100-HMG, apart from further proteins, belong.

During the reproductive age, the endometrium in the cavum uteri is subject to cyclic changes which are induced by hormones of the ovary. If endometrium accumulations are present at sites other than the interior of the uterus, this is referred to as endometriosis. Endometriosis occurs in approximately 10-15% of women of sexually mature age (approximately 20-40 years old). Because of symptomatology such as sterility, severe pain, bleeding and increased rate of miscarriage, endometriosis is of extremely important clinical relevance (Baltzer, J. and Mickan, H. (1994): Gynäkologie—Ein kurzgefasstes Lehrbuch (Gynaecology—an abridged textbook), Thieme, Stuttgart, page 206-214) (Gogusev, J. Bouquet de Joliniere, J., Telvi, L., Doussau, M., du Manior, S., Stoikoski, A., Levardon M, (1999): Detection of DNA copy number changes in human endometriosis by comparative genomic hybridisation. Hum Genet. 105: 444-451). Possible therapies are based on therapies for changing the hormone balance with hormone antagonists and on surgical interventions (laparoscopy or laparotomy) for the removal of endometriotic accumulations while retaining organs as far as possible (Schmidt-Matthiesen, H. and Hepp, H. (1998): Gynäkologie and Geburtshilfe (Gynaecology and obstetrics), Schattauer, Stuttgart, page 339-340). A disadvantage in the case of the therapy possibilities described above is that none of these therapies is causal.

The present invention is consequently based on the task of providing agents which permit a causal therapy of diseases belonging to the range of forms of endometriosis. Moreover, it is a task on which the present invention is based to provide an agent which permits the development of medicaments for the treatment of endometriosis and, in general, diseases of the endometrium. Finally, it is a task of the present invention to provide agents for diagnosing diseases of the endometrium. Moreover, the invention is based on the task of providing a contraceptive agent and/or a process for its production.

According to the invention, the task is achieved in a first aspect by the use of HMGB and/or a nucleic acid that codes therefor and/or an interaction partner of HMGB that is in particular natural and/or a nucleic acid that codes therefor as target molecule for the development and/or production of a medicament for the treatment and/or prevention of diseases of the endometrium and/or for the development and/or production of a diagnostic agent for diagnosing diseases of the endometrium.

In a second aspect, the task is achieved by the use of HMGB, a nucleic acid that codes therefor or an interaction partner that is in particular natural and/or a nucleic acid that codes therefor as target molecule for the development and/or the production of a medicament, the medicament being a contraceptive.

In an embodiment of the first and second aspect of the present invention, it is anticipated that the medicament comprises an agent which is selected from the group which comprises antibodies, peptides, anticalins, small molecules, antisense molecules, aptamers, Spiegelmers and RNAi molecules.

In a further embodiment it is anticipated that the agent enters into interaction with HMGB or with an interaction partner of HMGB that is in particular natural.

In a further embodiment it is anticipated that the agent enters into interaction with a nucleic acid that codes for HMGB and/or with a nucleic acid that codes for an interaction partner of HMGB that is in particular natural, in particular with mRNA, genomic nucleic acid or cDNA for HMGB.

In a third aspect, the task is achieved by the use of a polypeptide that enters into interaction with HMGB or with an interaction partner of HMGB that is in particular natural, for the production or development of a medicament, the medicament being one that is selected from the group which comprises medicaments for the treatment and/or prevention of diseases of the endometrium and contraceptives, and/or for the production or development of a diagnostic agent for diagnosing diseases of the endometrium.

In one embodiment it is anticipated that the polypeptide is selected from the group which comprises antibodies against HMGB and HMGB-binding polypeptides.

In a fourth aspect, the task is achieved by the use of a nucleic acid that enters into interaction with HMGB or with an interaction partner of HMGB that is in particular natural, for the production or development of a medicament, the medicament being one that is selected from the group which comprises medicaments for the treatment and/or prevention of diseases of the endometrium and contraceptives, and/or for the production or development of a diagnostic agent for diagnosing diseases of the endometrium.

In one embodiment, it is anticipated that the nucleic acid is selected from the group which comprises aptamers and Spiegelmers.

In a fifth aspect, the task is achieved by the use of a nucleic acid that enters into interaction with a nucleic acid that codes for HMGB or for an interaction partner of HMGB that is in particular natural, for the production or development of a medicament, the medicament being one that is selected from the group which comprises medicaments for the treatment and/or prevention of diseases of the endometrium and contraceptives, and/or for the production or development of a diagnostic agent for diagnosing diseases of the endometrium.

In one embodiment it is anticipated that the nucleic acid entering into interaction is an antisense oligonucleotide, ribozyme and/or RNAi.

In a further embodiment it is anticipated that the nucleic acid that codes for HMGB or for an interaction partner of HMGB that is in particular natural is the cDNA or mRNA concerned.

In an embodiment of all the aspects of the present invention it is anticipated that the disease of the endometrium is selected from the group which comprises endometriosis, endometrium polyps, hyperplasias of the endometrium and endometrium carcinoma.

In a further embodiment of all the aspects of the present invention it is anticipated that HMGB is selected from the group which comprises HMGB1, HMGB2, HMGB3 and SP100-HMG.

In a further embodiment of all the aspects of the present invention it is anticipated that the interaction partner of HMGB is RAGE (receptor for advanced glycation end products).

In a sixth aspect, the task is achieved by a pharmaceutical composition comprising at least one agent which is selected from the group which comprises polypeptides that enter into interaction with HMGB, with a nucleic acid that codes therefor, with an interaction partner of HMGB that is in particular natural and/or with a nucleic acid that codes therefor, nucleic acids that enter into interaction with HMGB or an interaction partner of HMGB that is in particular natural and nucleic acids that enter into interaction with nucleic acid(s) that codes or code for HMGB or for an interaction partner of HMGB that is in particular natural, and at least one pharmaceutically acceptable carrier, in particular for the treatment and/or prevention of diseases of the endometrium and/or for contraception.

In a seventh aspect, the task is achieved by a kit for the characterisation of the endometrial state, in particular for the determination of the presence of a pregnancy or cycle interferences, comprising a polypeptide that enters into interaction with HMGB, with a nucleic acid that codes therefor, with an interaction partner of HMGB that is in particular natural or with a nucleic acid that codes therefor, a nucleic acid that enter into interaction with HMGB, with a nucleic acid that codes therefor, with an interaction partner of HMGB that is in particular natural or with a nucleic acid that codes therefore and/or a nucleic acid that enters into interaction with a nucleic acid that codes for HMGB or for an interaction partner of HMGB that is in particular natural.

In an eighth aspect, the task is achieved by the use of RAGE or a derivative thereof for the development and/or production of medicament for the treatment and/or prevention of diseases of the endometrium and/or for the development and/or production of a diagnostic agent for diagnosing diseases of the endometrium.

In a preferred embodiment it is anticipated that the RAGE derivative is an sRAGE or a translation product of a nucleic acid according to SEQ IS NO. 4, 5 or 6.

The present invention is based on the surprising finding that the opinion previously widely held in the state of the art that the HMBG proteins such as HMGB1, HMGB2, HMGB3 and SP100-HMG, and HMGB1 in particular, are present ubiquitously in all cells in the same concentration is incorrect. Instead, the present inventors have found that the endometrium shows a particularly strong expression of HMGB, and HMGB1 in particular. In addition, the present inventors have found that the titer of HMGB, and HMGB1 in particular, changes significantly during the endometrial cycle. Finally, the present inventors have found that the quantity and/or concentration of RAGE which is an interaction partner of HMG, and HMGB1 in particular, also changes during the endometrial cycle. Without wishing to be committed to this in the following, this provides the possibility of preventing the effect of HMGB1 normally observed by blocking HMGB1 and/or its receptor. Prevention of the effects can be correspondingly exploited in, a therapeutic concept in the case of which the effects mediated by HMGB, HMBG1 in particular, and its interaction partner, in particular RAGE, are prevented. This can be advantageously exploited, e.g. for the prevention and treatment, in connection with the diseases of the entrometrium described herein. In addition, the HMG proteins and their interactions partner, in particular HMGB1 and RAGE, are thus suitable labels for monitoring the state of the endometrium and/or of diseases of the endometrium as disclosed herein. In this connection, the disease concerned is diagnosed, therapeutically treated, influenced in its progression, preferably slowed down and/or the therapy success monitored preferably by reducing the biologically active HMGB and/or its interaction partner such as, in particular, RAGE.

These surprising insights into the significance of HMGB and its interaction partners, in particular RAGE, in the endometrial events form the basis for the various aspects of the present invention.

The term HMGB should be understood to mean in this case all proteins of the HMGB family. In this respect, the present invention relates to the use of the HMGB proteins and the nucleic acids that code for them. In particular, the invention relates in this respect in its different aspects disclosed herein to the use of HMGB1, HMGB2, HMGB3 and SP100-HMG and in particular HMGB1 and/or the nucleic acids that code for them. HMGB should be understood to include also those HMGB molecules exhibiting deletions, mutations and modifications, for example, which are referred to herein in general terms as modified HMGB proteins. An HMGB protein according to the meaning of the present invention is regarded as being an HMGB protein for as long as it exhibits or possesses at least one of the properties of the non-modified form. Corresponding modifications are within the ability of the persons skilled in the art in this field.

The present invention will be explained in the following by way of HMGB1 as an example.

HMGB1 belongs to the group of high mobility group proteins. HMGB1 is the protein of the HMGB family that has been investigated most thoroughly. Basically, two different functions of HMGB1 are known. On the one hand, being a so-called architectonic transcription factor, it influences the formation of transcription factor complexes in the cell nucleus for certain target genes such as e.g. the binding of the oestrogen receptor to specific DNA sequences (Boonyaratanakornkit, V., Melvin V., Prendergast, P., Altmann, M., Ronfani, L. Bianchi, M E., Tarasevicience, L., Nordeen, S K., Allegretto, E A., Edwards, D P (1998): High mobility group chromatin proteins 1 and 2 functionally interact with steroid hormone receptors to enhance their DANN binding in vitro and transcriptional activity in mammalian cells. Mol. Cell. Biol 18: 4471-4487). On the other hand, HMGB1 is an extracellular ligand of the cell surface receptor RAGE (receptor for advanced glycation end products) (Taguchi, A., Blood, D C., del Toro, G., Canet, A., Lee, D C., Qu, W., Tanji, N., Lu, Y., Lalla, E., Fu, C., Hofmann., M A., Kislinger, T., Ingram, M., Lu, A., Tanaka, H., Hori, O., Ogawa, page Stern, D M., Schmidt, A M. (2000): Blockade of RAGE-amphetorin signalling suppresses tumour growth and metastases. Nature 405: 354-360) and RAGE is consequently an interaction partner of HMGB according to the meaning of the present invention. In this function, HMGB1 participates in the tumour metastasization and plays a part in central cellular signal transfer paths such as e.g. p21ras, MAP kinase, NF-kB and cdc42/rac. The ability to secrete HMGB1 is restricted to only certain cell types: macrophagen, neurons and specific tumour cell lines (Taguchi, A., Blood, D C., del Toro, G., Canet, A., Lee, F C., Qu, W., Tanji, N., Lu, Y., Lalla, E., Fu, C., Hofmann., M A., Kislinger, T., Ingram, M., Lu, A., Tanaka, H., Hori, O., Ogawa, page Stern, D M., Schmidt, A M. (2000): Blockade of RAGE-amphetorin signalling suppresses tumour growth and metastases. Nature 405: 354-360) and (Miller, S., Scaffidi, P., Degryse, B., Bonaldi, T., Ronfani, L., Agresti, A., Beltrame, M., Bianchi, M E., (2001): New EMBO members' review: the double life of HMGB1 chromatin protein: architectural factor and extracellular signal. EMBO J. 20: 4337-4340).

Endometriosis is a distinctly complex process the pathogenesis of which is still unclear. Histologically, endometrial glands emerge which are surrounded by stroma (Thomas, C. (1998): Histopathologie. Schattauer, Stuttgart, page 249-250). Consequently, a tissue modification is involved which consists both of epithelium and mesenchyma. According to valid opinion, endometriosis is a mucosal ectopy.

Moreover, depending on its position, endometriosis is divided into different groups (Baltzer, J. and Mickan, H. (1994):Gynäkoloqie—Ein kurzgefasstes Lehrbuch (Gynaecology—an abridged textbook), Thieme, Stuttgart, page 206-214) which are referred to here in general terms as endometriosis:
1. Endometriosis genitalis interna, mainly on the uterus wall (Adenomyosis uteri) and tuba
2. Endometriosis genitalis externa, mainly retrocervical Douglas peritoneum, ovary and portio
3. Endometriosis extragenitalis, mainly the bladder, surgical scars, pleura, ureter and intestine.

These ectopic mucosal islands participate in the cycle to a large extend under the influence of ovarian hormones and consequently exhibit periodic changes, i.e. mucous membrane proliferation, indications of the secretory phase, premenstrual edema and mucosal degradation as well as discharge of blood.

The changes taking place during this process result in typical pain, depending on the location and the extent of the accumulations. Moreover, a premenstrual onset of pain and a decrease in menstruation are typical. As a result of the lack of possibilities for discharge of the degrading tissue and the bleeding, cystic distension of the organs and adhesions may occur which in turn may lead to permanent pain USCHMIDT-MATTHIESEN, H. AND HEPP, H. (1998): Gynäkologie and Geburtshilfe (Gynaecology and obstetrics), Schattauer, Stuttgart, page 339-340).

Numerous different hypotheses exist regarding the pathogenesis of endometriosis (Baltzer, J. and Mickan, H. (1994): Gynäkologie—Ein kurzgefasstes Lehrbuch (Gynaecology—an abridged textbook), Thieme, Stuttgart, page 206-214) (Scheider, A. (2001): Frauenheilkunde. Auszug aus der Hauptvorlesung (Gynaecology. Excerpt from the main lecture). University of Jena. www.unijena.de/ufk/cd/endomose.htm), among which those with the greatest plausibility are the following although at present a final assessment does not appear to be possible and possibly several factors may play a part in the pathogenesis:
“de novo” (spreading of endometrium tissue to non-local sites). The spreading may again occur in different ways, namely by retrograde menstruation, vascular proliferation, mechanical spreading. (by surgical interventions), by invasion of the endometrium into the myometrium, by suction as a result of preovulatory utero-tubal peristalsis (chocolate cysts may form).
“in situ” (conversion of multicentric epithelium into endometrium tissue): embryonal residual tissue (coelomic epithelium, epithelium from remainders of the mullerian canals and/or the woiffian canals) is transformed by metaplasia into endometrium tissue.
endometrium-induced metaplasia: endometrium parts reaching the peritoneum as a result of retrograde menstruation cause a differentiation of the non-differentiated mesenchymal cells into endometrial tissue.

As a result of the increased expression, disclosed herein, of HMBG and, of HMGB1 in particular, in endometrial tissue, it is possible to distance oneself from the opinion prevailing in the state of the art so far that the HMGB proteins, the nucleic acid that codes for them, but also the interaction partners, in particular the natural interaction partners, of HMGB and HMGB1 in particular do no represent target molecules or targets which can be used for the development of medicaments. In the light of the disclosure made herein, however, these are rather target molecules relevant to the highest degree for the production or development of a medicament or a diagnostic concerning endometric tissue and specifically of diseases of the endometrium. Diseases of the endometrium, as the term is used here, relates in particular to endometriosis in the different embodiments described herein but also to endometrium polyps, hyperplasias of the endometrium and endometrium carcinoma.

The use of the HMGB proteins, the nucleic acids that code for them, the HMGB interaction partners, in particular the natural HMGB interaction partners, and/or the nucleic acids that code for them as target can be used in this respect in connection with any production and/or development process for a medicament and/or diagnostic agent.

The medicament or the diagnostic agent can in this respect be an antibody, an HMGB1-binding peptide, an HMGB1-binding anticalin, a small molecule entering into interaction with HMGB1, a nucleic acid entering, into interaction with HMGB1 such as e.g. an aptamer or Spiegelmer or again a nucleic acid that enters into interaction with a nucleic acid that codes for HMGB1, e.g. mRNA or cDNA. The medicament or the diagnostic agent can in this case also be a truncated and/or soluble form of RAGE, the so-called sRAGE, an antibody to RAGE, a RAGE-binding peptide, a RAGE-binding anticalin, a small molecule entering into interaction with RAGE, a nucleic acid entering into interaction with RAGE such as e.g. an aptamer or a Spiegelmer or a nucleic acid that enters into interaction with a nucleic acid that codes for RAGE, e.g. mRNA or cDNA, the medicament and/or the diagnostic agent being suitable for use in any case for the treatment, prevention and/or diagnosis of different diseases as disclosed herein.

In this connection it is within the framework of the present invention that, instead of RAGE, as it occurs naturally, be it of human or other origin, in particular other mammalian sources, truncated forms and/or soluble forms thereof can also be used which are also known in the state of the art. Moreover, it is within the framework of the present invention that the abbreviated form of RAGE as it is described herein in the exemplary part and encoded by the nucleic acids according to SEQ ID NO. 4, 5 and 6, can be used within the framework of the present invention.

For the production of an antibody specific for HMGB1, processes known according to the state of the art are used such as those known to the persons skilled in the art in the field. Particularly preferred in this connection is the use of monoclonal antibodies which can be produced according to the protocol of Cäsar and Milstein, and further developments thereof. In this respect, antibodies are also antibody fragments or antibody derivatives such as e.g. Fab fragments, Fc fragments but also single-stranded antibodies, for as long as these are generally capable of specifically binding HMGB. Apart from monoclonal antibodies, polyclonal antibodies can also be used. One polyclonal antibody for basic research which could, in principle, also be used as a medicament is the antibody sc-12523 which is aimed e.g. against HMGB1 (Santa Cruz Biotechnology, Santa Cruz, USA). Preferably, the antibodies used are human or humanised antibodies. The details given above regarding HMGB1 apply in terms of their meaning also to HMGB, the interaction partners of HMGB, in particular HMGB1, for example. RAGE.

A further class of medicaments which could be produced as targets using the HMGB proteins, the nucleic acids that code for them, the HMGB interaction partners, in particular the natural HMGB interaction partners and/or the nucleic acids that code for them, are peptides binding to them. Such binding peptides can be produced in a screened manner using processes known in the art such as e.g. phage display. These techniques are known to those skilled in the art in this field. In this case, the procedure for producing such peptides is typically such that a peptide library is set up, e.g. in the form of phages, and this library is brought into contact with a target molecule, e.g. with HMGB1 in the present case. The binding peptides are then typically removed as a complex together with the target molecule from the non-binding members of the library. In this case, it is within the framework of the knowledge of the persons skilled in the art of this field that the binding properties depend, at least to a certain extent, from the test conditions present in concrete terms in each case such as e.g. the salt content and such like. After separating off the peptides binding with a higher or stronger affinity or force to the target molecule from the non-binding members of the library and/or from the target molecule, these can then be characterised. If necessary, an amplification step is required before the characterisation, e.g. by multiplying the corresponding phages that code for the peptide or peptides. The characterisation preferably comprises the sequencing of the peptides binding to HMGB1. In this respect, the peptides are, in principle, not restricted regarding their length. Typically, however, peptides with a length of 8 to 20 amino acids are obtained and/or used in such processes. The size of the libraries is 10² to 10¹⁸, preferably 10⁸ to 10¹⁵ different peptides. The details given above regarding HMGB1 apply in terms of their meaning also to HMGB, the interaction partners of HMGB, in particular HMGB1, such as RAGE.

A special form of polypeptides binding to target molecules consists of the anticalins, e.g. those described in German patent application DE 197 42 706.

When using the HMGB proteins, the nucleic acids that code for them, the HMGB interaction partners, in particular the natural interaction partners and/or the nucleic acids that code for them as target molecules for the manufacture and/or development of a medicament for the treatment of diseases of the endometrium as well as for the manufacture and/or development of agents for diagnosing endometriosis diseases, libraries of smaller molecules can also be used. In this case, too, the target molecule, i.e. one or several of the HMGB proteins or an interaction partner thereof such as RAGE, can be brought into contact individually or, if necessary in combination, with a library of smaller molecules and those members of the library which bind to it can be determined, if necessary separated off from the other members of the library and/or the target molecule, and optionally characterised further. The characterisation of the small molecule takes place according to procedures known to those skilled in the art in this field; in this way, it is e.g. possible to identify the compound and to determine the molecular structure. These libraries comprise as few as two and as many as up to several hundred thousand members.

It is also within the scope of the present invention for HMGB proteins, nucleic acids that code for them, HMGB interaction partners, in particular natural interaction partners and/or nucleic acids that code for them, to be used as target molecules for the production of aptamers and Spiegelmers, these then being used directly or indirectly as medicament.

Aptamers are D-nucleic acids which are either single-stranded or double-stranded and bind specifically to a target molecule. The production of aptamers has been described e.g. in European patent EP 0 533 838. The procedure is as follows:

In the process for the production of the aptamers, a mixture of nucleic acids, i.e. potential aptamers is provided, each nucleic acid consisting of a segment of at least eight successive randomised nucleotides and this mixture being brought into contact with the target molecule or target, in the present case therefor with HMGB proteins, nucleic acids that code for them, HMGB interaction partners, in particular the natural interaction partners and/or the nucleic acids that code for them, nucleic acids which bind to the target, possibly as a result of an increased affinity compared with the candidate mixture, being separated off from the remainder of the candidate mixture and the nucleic acids binding to the target, possibly with an increased affinity or force, which are thus obtained being amplified. These steps are repeated several times such that at the end of the process, nucleic acids binding specifically to the target or target molecule concerned, i.e. the so-called aptamers, are obtained. It is within the framework of the present invention that these aptamers can be stabilised, e.g. by introducing certain chemical groups, as is known to those skilled in the art in the field of aptamer development. At present, aptamers are already being used for therapeutic purposes. It is also within the framework of the present invention for aptamers produced in this way to be used for target validation and/or as guide substances for the development of medicaments, in particular of small molecules.

A basically similar principle forms the basis for the production or preparation of Spiegelmers which can be developed within the framework of the present invention on the basis of the HMGB proteins, the nucleic acids that code for them, the HMGB interaction partners, in particular the natural interaction partners and/or the nucleic acids that code for them, as target molecule. The production of Spiegelmers is described e.g. in the international patent application WO 98/08856. Spiegelmers are L-nucleic acid, consisting e.g. of L-nucleotides, and are essentially characterised by the fact that they have a very high stability in biological systems and, comparable to aptamers, are capable simultaneously of interacting specifically with a target molecule and/or binding to it. More precisely, the procedure in the production of Spiegelmers consists of producing a heterogeneous population of D-nucleic acids, bringing the population into contact with the optical antipodes of the target molecule, in the present case consequently with the D-enantiomer of the naturally occurring L-enantiomer, subsequently separating off those D-nucleic acids which have not entered into interaction with the optical antipode of the target molecule, determining, if necessary separating off and sequencing the D-nucleic acids which have entered into interaction with the antipode of the target molecule and subsequently synthesising L-nucleic acids which are identical in terms of their sequence to that of the sequences previously determined for the D-nucleic acids. Similar to the process for the production of aptamers, it is also possible in this case to enrich and/or produce suitable nucleic acids, i.e. Spiegelmers, by repeatedly repeating the steps.

A further class of compounds which can produced and/or developed using HMGB proteins and interaction partners thereof and/or the nucleic acids that code for them are the ribozymes, antisense oligonucleotides and RNAi.

All these classes have the common feature that they are unable to deploy their effect at the level of the translation production, i.e. at the level of the proteins (HMGB proteins and protein interaction partners thereof) but at the level of the nucleic acid; that code for the protein concerned, in particular the mRNAs that code for HMGB1.

Ribozymes are the catalytically active nucleic acids which are preferably built up of RNA and consist of two partial regions. The first partial region is responsible for a catalytic activity, whereas the second part is responsible for a specific interaction with the target nucleic acid. If an interaction between the target nucleic acid and the second part of the ribozyme takes place typically by hybridisation of two base regions which are essentially complementary to each other, the catalytic part of the ribozyme may hydrolyse the target nucleic acid either intramolecularly or intermolecularly, the latter being preferred, in the case that the catalytic effect of the ribozyme is a phosphodiesterase activity. Subsequently, a—possibly further—degradation of the encoding nucleic acid takes place, the titer of the target molecule being reduced both at the level of the nucleic acid and the level of the protein both intracellularly and extracellularly, a therapeutic approach being thus provided in the case of diseases of the endometrium. Ribozymes, their use and construction principles are known to those skilled in the art in this field and have been described e.g. by Doherty and Doudna (Ribozyme structures and mechanics. Annu Rev Biophys Biomol Struct 2001; 30:457-75) and Lewin and Hauswirth (Ribozyme gene therapy: applications for molecular medicine. Trends Mol Med 2001, 7:221-8).

A basically similar mechanism of action forms the basis for the use of antisense oligonucleotides for the production of a medicament and/or diagnostic agent. Antisense oligonucleotides hybridise as a result of base complementarity typically with a target RNA, normally with mRNA, and as a result activate RNAaseH. RNAaseH is activated both by phosphodiesters and phosphorothioate-coupled DNA. However, phosphodiester-coupled DNA is rapidly degraded by cellular nucleases, with the exception of phosphorothiate-coupled DNA. These resistant DNA derivatives occurring in a non-natural way do not inhibit RNAaseH if they are hybridised with RNA. In other words, antisense polynucleotides are effective only as a DNA-RNA hybrid complex. Examples of such antisense oligonucleotides can be found in the US patent specification U.S. Pat. No. 5,849,902 or U.S. Pat. No. 5,989,912 among others. Basically, the essential concept of the antisense oligonucleotides consists of providing a complementary nucleic acid against certain RNAs. In other words, starting out from the knowledge of the nucleic acid sequence of the HMGB proteins and/or their interaction partners, in particular the mRNA concerned, suitable antisense oligonucleotides can be produced by base complementarity which oligonucleotides lead to degradation of the encoding nucleic acid, in particular mRNA.

A further class of compound which would basically be suitable as medicament and/or diagnostic agent and in particular also for the therapy and/or prevention and/or diagnosis of the diseases of the endometrium described herein is the so-called RNAi. RNAi is a double stranded RNA which provides RNA interference and typically has a length of approximately 21 to 23 nucleotides. In this respect; one of the two strands of the RNA corresponds to a sequence of a gene to be degraded. In other words, starting out from the knowledge of the nucleic acid that codes for HMGB and/or its interaction partner, in particular mRNA, a double stranded RNA can be produced, one of the two RNA strands being complementary to the said nucleic acid that codes for HMGB and/or its interaction partner, preferably. mRNA and this then leads to the degradation of the corresponding encoding nucleic acid and, simultaneously, to a reduction of the titer of the proteins concerned. The production and use of RNAi as medicament or diagnostic agent has been described in the international patent applications WO 00/44895 and WO 01/75164, for example.

With a view to the mechanism of action of the classes, ribozymes, antisense oligonucleotides and RNAi described above, it is consequently within the framework of the present invention to use, apart from the HMGB proteins and their interaction partners that are in particular natural, also the nucleic acids that code therefor, in particular mRNA, for the peroduction of a medicament for the treatments and/or prevention of diseases of the endometrium and/or for the production of a diagnostic agent for diagnosing of endometric diseases and monitoring of the course of the disease and/or the therapy applied, either directly or as a target molecule.

It is, moreover, within the framework of the present invention that the classes of compounds mentioned above, i.e. antibodies, peptides, anticalins, small molecules, aptamers, Spiegelmers, ribozymes, antisense oligonucleotides and RNAi can be used for the production of a medicament for the treatment of diseases of the endometrium and/or for the production of a contraceptive. The compounds of the different classes produced in this way can also be the subject matter of a pharmaceutical composition or a diagnostic agent which is preferably used for the treatment of diseases of the endometrium and/or as a contraceptive. In one embodiment, the pharmaceutical composition comprises, apart from one or several of the above-mentioned and/or produced compounds as disclosed herein, also other pharmaceutically active compounds such as steroid hormones and a pharmaceutically acceptable carrier. Such carriers can be e.g. liquid or solid, e.g. a solution, a buffer, an alcoholic solution and such like. Starch, for example, and such like can be considered for use as suitable solid carriers. The persons skilled in the art in the field of pharmaceutical administration forms are aware how the corresponding compounds of the different classes need to be formulated in order to be able to be administered in the desired form of administration, e.g. orally, parenterally, subcutaneously, intravenously and such like.

The different compounds of the different classes can also be individually or jointly the object of a kit, the kit comprising the compound and, if necessary, one or several other elements which are selected from the group which comprises buffers, negative controls, positive controls and instructions for use. Typically, the individual compounds are contained in the kit in the dry or liquid form, preferably in portions for the individual cases of application. In this respect, the kit can preferably used for the determination of the state of the endometrium on the basis of the correlation, disclosed herein, between the titer of HMGB proteins and HMGB1 in particular and the course of the endometrial cycle over time. A particular application is the use of the kit for the determination of the presence of a pregnancy and/or cycle disturbances. This is based on the observation disclosed herein that differences in the HMGB expression, and especially the HMGB1 expression, may occur in the cycle phase.

In connection with the different classes of compounds disclosed herein which can be used according to the invention as therapeutic agent or diagnostic agent, one aspect is that certain classes enter into interaction directly with the HMGB proteins and/or the interaction partner(s) of the HMGB proteins which are present in particular as protein(s). However, it is also within the framework of the present invention that the said compounds of the different classes, in particular if they involve peptides, antibodies, aptamers and Spiegelmers, block the interaction partner of the HMGB by a more or less specific interaction for the HMGB. To this extent, the concept of the use of HMGB herein should also be understood to mean that it comprises the use of one or several of the HMGB interaction partner(s) such as receptors, for example. The processes described herein then need to be modified insofar that, instead of the HMGB proteins and/or the nucleic acids that code for them, an interaction partner thereof is used in the different selection processes, assays, screening processes or production processes.

Without wishing to be committed thereto in the following, the reason for the effectiveness of active substances directed against HMGB and HMGB1 in particular or their interaction partner(s) seems to be based on the prevention of the formation of transcription factor complexes and/or in the case of those disease of the endometrium in the case of which HMGB1 in particular is present as an extracellular ligand, to block its interaction with its target structures or the target structures themselves. The same applies also to the use of the classes of compounds, disclosed herein, for the production of a contraceptive, it being used as a starting point that HMG proteins, HMGB1 in particular, participate in the building up of the endometrium and the capture or blocking of the HMGB proteins and/or their effect lead to no building up of the endometrium occurring and consequently the preconditions necessary for a pregnancy or an implantation of the fertilised ovum are not fulfilled, as a result of which a pregnancy is prevented.

Within the framework of the present invention, natural interaction partners of the HMGB proteins, which are generally referred to as HMGB herein, refers to those molecules and structures with which these enter into interaction. In particular, those molecules and structures are involved with which the proteins in the biological system enter into interaction under normal but also under pathological conditions. These include, among others, receptors and those molecules and structures which participate in the formation of transcription factor complexes in which HMGB proteins also participate. An example of an interaction partner for HMGB proteins is RAGE, as described herein.

Incidentally, the terms protein, polypeptide and peptide are used as synonyms herein, unless the contrary is indicated.

Finally, it is within the framework of the present invention that it relates to all those compounds which can be produced by the processes described herein or can be obtained within the framework of the different screening processes described.

For the group of HMGB proteins disclosed herein which comprises HMGB1, HMGB2, HMGB3 and SP100-HMG, the specific results regarding HMGB1 apply in particular to the extent that a very high homology exists between the different compounds.

The invention will now be explained by way of the following figures and examples from which further characteristics, embodiments and advantages of the invention result. In this respect

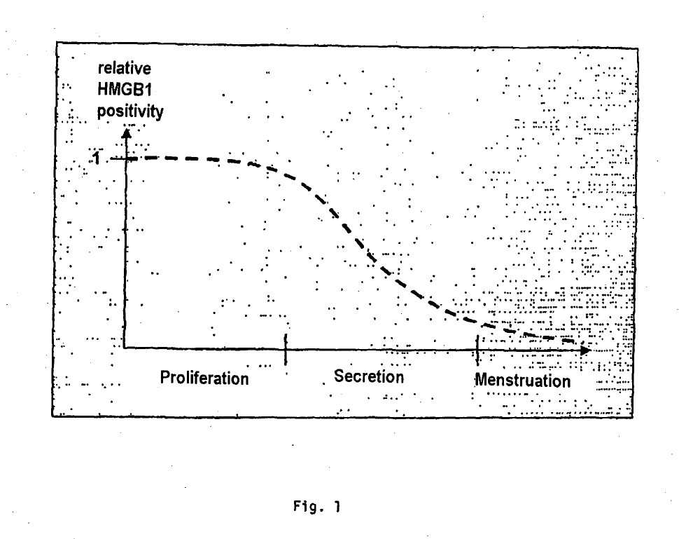

FIG. 1 shows the connection between the HMGB1 positivity in the endometrial cycle over time.

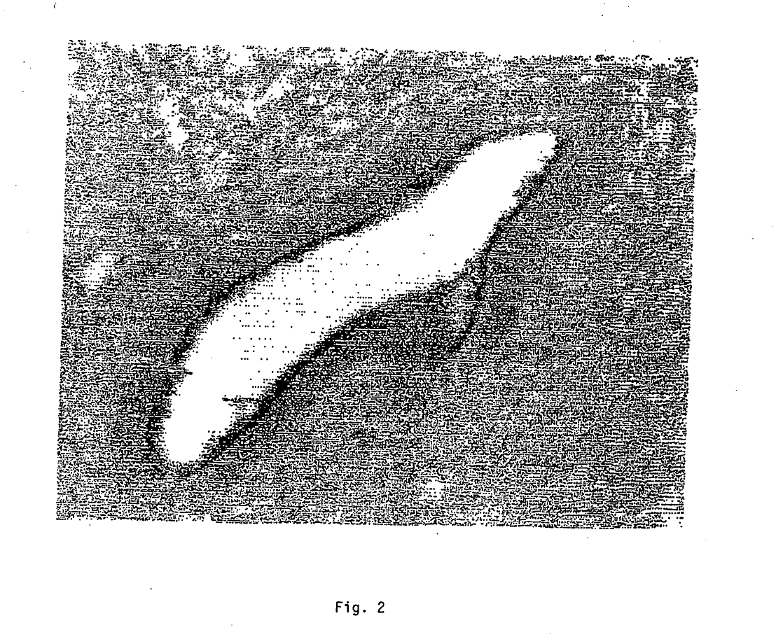

FIG. 2 shows an immunohistochemical illustration of an endometriosis area using an HMGB1-specific antibody.

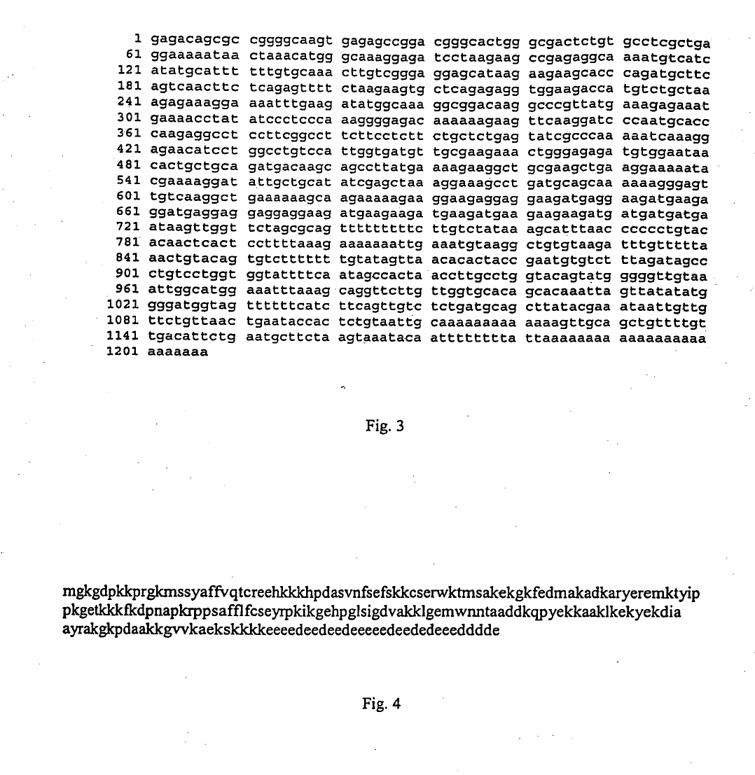

FIG. 3 shows the cDNA sequence of the HMGB1 gene, the protein-coding sequence beginning at position 77 and ending, at position 724 (accession number BC003378)

FIG. 4 shows the amino acid sequence of the HMGB1 protein (accession SO2826) and

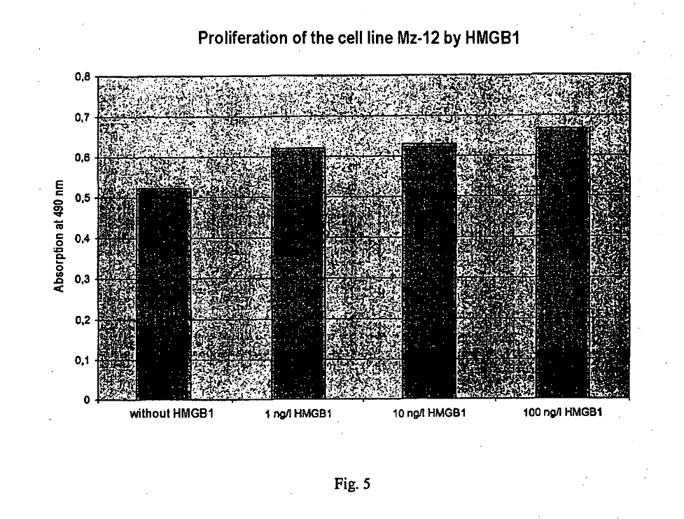

FIG. 5 shows a bar chart which illustrates the proliferation rate of the endometrium carcinoma cell line Mz-12 following the application of different HMGB1 concentrations.

### EXAMPLE 1

### The Expression of the HMGB1 Protein as a Function of Different Cycle Phases

Immunohistochemical investigations were carried out on paraffin sections (5 μm) of human uterus endometria and endometrioses were carried out with a polyclonal antibody (sc-12523, Santa Cruz Biotechnology, Santa Cruz, USA) from the goat which is directed against the peptide from an inner region of the human HMGB1 protein. The antibody used detects the HMGB1 protein, and to a lesser extent, the HMGB2 protein.

The surprising result of the immunohistochemical investigation of a total of 25 tissue sections with a normal endometrium and an adjacent myometrium in different cycle phases consists of the fact that the HMGB1 protein could be detected specifically only in the cytoplasm membrane of the gland epithelium in the proliferation phase of the menstrual cycle. In the secretion phases, the positivity of HMGB1 decreases overall in the cytoplasm membrane but is still detectable in some cases (FIG. 1). The connective tissue cells of the endometrium and the smooth muscle cells of the myometrium did not exhibit any immune response in any cycle phase.

### EXAMPLE 2

### Immunohistochemical Detection of Endometriosis Accumulations

Immunohistochemical investigations were carried out on 20 endometriosis preparations using the HMGB1 antibody and it was found that endometrium accumulations surprisingly enough exhibit a strong positivity in the cytoplasm membrane of the gland epithelium but no HMGB1 positivity in the other cell compartments and the surrounding tissue. As an example, a result of the investigation is illustrated in FIG. 2 which shows a tissue section of an adenomyosis following HMGB1-specific immunohistochemistry. A section of an adenomyosis (Adenomyosis uteri) is illustrated 400 times enlarged.

### EXAMPLE 3

### Increased Cell Proliferation as a Result of The Application of HMGB1 In Vitro

### Methodology:

The proliferation rate was measured using the CellTiter 96® AQᵤₑₒᵤₛ one solution cell proliferation assay from Promega. In this case, the biological reduction potential of vital cells is determined by colorimetric measurement.

In preparation, cells of the endometrium carcinoma cell line Mz12 were cultivated in RPMI 1640 medium with 10% fetal calf serum at 37° C. and with 5% CO₂ until the cells reached a confluent density. Following trypsinisation of the cells, these were absorbed in serum-free RPMI 1640 medium and uniformly distributed over plates of 96 wells with 100 μl RPMI 1640 medium (without fetal calf serum) per well and incubated overnight at 37° C. and with gasification by 5% CO₂. After sucking off the medium, 100 μl of a mixture, formed from a dilution series, of medium and HMGB1 protein with a defined concentration of the HMGB1 protein (1 ng/l, 10 ng/1 and 100 ng/1 HMGB1) per well were placed on the Mz12 cells. Following 24 h incubation at 37° C. and gasification with 5% CO₂, 20 μl cellTiter 96® AQᵤₑₒᵤₛ one solution per well were added. The evaluation for the determination of the proliferation rate took place after 1h by measuring the absorption at 490 nm using an Anthos reader, model 2001, from Anthos.

### Results:

In comparison with the cells of the negative control, i.e. without HMGB1 application, it was possible to determine a significant increase in the proliferation rate in the case of the cells treated with HMGB1. Moreover, the increase in the proliferation rate correlates with the HMGB1 concentration, as shown in FIG. 5.

### EXAMPLE 4

### Labelling of HMGB1 with Fluorescein

### Methodology:

Labelling of HMGB1 was carried out using the fluorescein labelling kit from Roche. 100 μg of HMGB1 protein were lyophilised in each batch and resuspended in 100 μl of PBS buffer. 1.5 μl of FLUOS solution (20 mg/ml) were added to the dissolved HMGB1, FLUOS being the fluorescent dye of the kit for labelling proteins, and the free amino group of the protein to be labelled was reacted with the 5(6) carboxyfluorescein-N-hydroxysuccinimide ester to give a stable amide bond, and the batch was incubated with stirring for 2 h at room temperature while being protected from the light.

In the meantime, the Sephadex-G-25 column was equilibrated with 5 ml of blocking solution which, like PBS, is a component of the kit concerned and is prepared initially in powder form as a blocking agent and then with doubly distilled water, in line with the instructions, as well as ml of PBS. Subsequently, the reaction batch was transferred to the column and the labelled protein eluted with 3.5 ml of PBS. The labelled protein was contained in each case in the first two pools of 10 drops (approximately 0.5 ml) each.

### Results:

A verification of the labelling took place in reversed phase HPLC using a C18 column and a binary gradient by determining the retention time (comparison of the peaks of FLUOS solution/labelled HMGB1). In addition, the labelled HMGB1 was separated into a 12% PA gel under denaturisation conditions. During this process, the labelled protein exhibited a distinct band in UV light which band could be confirmed by Coomassie colouration. Approximately 60 μg of HMGB1 were labelled in an end volume of 1 ml using fluorescein.

### EXAMPLE 5

### Binding of Fluorescein-Labelled HMGB1 Protein to the Cell Membrane of Cells of the Endometrium Carcinoma Cell Line Mz-12

### Methodology:

In preparation, the Mz-12 cells were incubated in Leighton tubes, i.e. special tubes for cultivating cells, using 1 ml of RPMI 1640 medium in each case overnight at 37° C. and with 5% CO₂. Subsequently, 350 μl of PBS and 6 μg of labelled HMGB1 protein prepared as in example 4 and 6μ of FLUOS solution used as negative control were added to the Mz-12 cells. After 1 h 30 min incubation at 37° C. and with 5% CO₂, the cover glasses were briefly washed in PBS and subsequently covered on a slide. The evaluation took place after approximately 2 h.

### Results:

After an incubation time of 2 h, membrane labelling of the Mz-12 cells by binding fluorescein-labelled HMGB1 proteins to the cell membrane was observed.

The use of the pure fluorescein did not result in a membrane positivity but merely in a greenish diffuse colouration of the cytoplasm.

### EXAMPLE 6

### Cloning of 3 Different RAGE Transcripts

### Methodology

The PCR amplificates of the endometrium carcinoma cell line (example 7) were separated in a 1.2% agarose gel and cut out from the gel using a sterile scalpel. The elution of the DNA took place by means of the QIAEX II system from Qiagen. The eluted DNA was ligated according to the “Ligations Using the pGEM®-T and pGEM®-T Easy Vectors and the 2× Rapid Ligation Buffer” protocol from Promega using T4 DNA ligase into the pGEM®-T Easy vector system and transformed in DH5a E. coli using the method of INOUE et al (1990). The isolation of the plasmid DNA took place according to the “QIAprep® Miniprep” handbook according to “QIAprep Spin Miniprep Kit Protocol” from QIAGEN. For sequencing, 3 μg of DNA from the plasmid DNA were evaporated in a SpeedVac. Sequencing with the primers M13 uni (5′-CCCAGTCACGACGTTGTAAAACG-3′) (SEQ ID NO. 7) and M13 rev (5′-AGCGGATAACAATTTCACACAGG-3′) (SEQ ID NO. 8) took place using the ABI 377 DNA sequencer (PE-Applied Biosystems, Weiterstadt).

The sequences of the plasmid DNA were processed using the EditSeq, MegAlign and Seqman (DNAstar) computer program. The comparison of the sequences with sequences that were already known took place by using the BLAST Service of the National Centre for Biotechnology Information (NCBI, USA) using the BLAST program (ALTSCHUL ET AL, 1990). Also, the sequences were examined for all repetitive sequencing using the RepeatMasker program.

### Results:

Following PCR and gel electrophoresis, it was possible to detect, besides the RAGE band in each case which corresponded to the expected product length of the RAGE transcript, three further bands of different intensity which, after cloning and sequencing, proved to be RAGE specific and were consequently referred to as sRAGE1, sRAGE2 and sRAGE3.

The 653 by fragment was referred to sRAGE1. Basically, it corresponds to the sequence of the RAGE fragment; in addition, however, 142 by insertion of the complete intron 6 of the RAGE gene took place between exons 6 and 7 of the transcript as well as the loss of exon 10 which was replaced by the first 82 by of intron 9 of the RAGE gene. As a result of the intron 6 insertion, 20 new amino acids are encoded (GEHRWGGPQAHVSTFWKSDP.) following the last amino acid (tryptophan) completely encoded by exon 6, including a newly generated stop codon. The new stop codon leads to a loss, at the protein level, of the extracellular C′ domain, the transmembrane domain and the cytosol domain of the RAGE receptor.

The 511 by sRAGE2 fragment corresponds, in principle, to the sequence of the RAGE fragment; however, exon 10 is replaced by the first 82 by of intron 9 of the gene. As a result, 17 new amino acids (GEGFDKVREADSPQHM) are encoded following the last complete amino acid (alanin) encoded by exon 9, including a newly generated stop codon. At the protein level, this leads to a loss of transmembrane domain and cytosol domain of the RAGE receptor.

The 698 by sRAGE3 fragment corresponds, in principle, to the sequence of the RAGE fragment; however, additionally, a 142 bp insertion of the complete intron 6 of the RAGE gene takes place between exons 6 and 7 of the transcript. As a result, 20 new amino acids (GEHRWGGPQAHVSTFWKSDP) are encoded following the last complete amino acid (tryptophan) encoded by exon 6, including a newly generated stop codon. At the protein level, the new stop codon leads to a loss of transmembrane domain and cytosol domain of the RAGE receptor.

### EXAMPLE 7

### RT-PCR for the Detection of the Rage Receptor

### Methodology:

Tissue specimens of the endometrium and myometrium were frozen in liquid nitrogen directly after removal. The RNA isolation from the tissue and the cells of the endometrium carcinoma cell line Mz 12 took place according to the RNeasy Mini Handbook from Qiagen. During this process, the RNA is bound to specific columns and purified by specific wash steps.

The cDNA of the total RNA was synthesised using the adaption primer AP2 (5′-AAGGATCCGTCGACATC(T)₁₇₋3′) (SEQ ID NO. 9) and M-MLV reverse transcriptase (Invitrogen, Life Technologies). The detection of the RAGE gene takes places using the primer RAGE2 up (5′-GATCCCCGTCCCACCTTCTCCTGTAGC-3′) (SEQ ID NO. 10) and RAGE2 lo (5′-CACGCTCCTCCTCTTCCTCCTGGTTTTCTG-3′) (SEQ ID NO. 11) in 0.2 ml cups and a 20 μl reaction volume. 125 ng cDNA were used as template. For the reaction, 0.5 μl of the recombinant Taq polymerase (5 U/μl) (Quiagen, Hilden Germany) and 2 μl of Quiagen PCR buffer (10×) were used. 4 μl of 5×Q solution (Qiagen), 2 μl of dATP, dGTP, dCTP, dTTP, (2 mM); and 0.4 μl of pro primer (10 μM) were added to the batch. The DNA amplification took place in a gradient thermocycler from Eppendorf for 35 cycles under the following conditions: 30 sec at 94° C., 30 sec at 69° C., and 1 min at 72° C. The initial denaturisation took place for 2 minutes at. 95° C. and a final elongation for 10 minutes at 72° C.

### Results:

Following gel electrophoretic separation, the RT-PCR showed, both with 10 myometrium tissues and 10 endometrium tissues and with the endometrium carcinoma cell line, a distinct specific band 556 bp in size which corresponded to the expected product length of the RAGE transcript. In addition, three further bands of different intensity could be detected which, after cloning and sequencing, proved to be RAGE specific (example 6).

### Sequences:

### Sequences of the Rage Transcripts

sRAGE1 (653 bp)

(SEQ ID NO. 4)

GATCCCCGTCCCACCTTCTCCTGTAGCTTCAGCCCAGGCCTTCCCCGACA
CCGGGCCTTGCGCACAGCCCCCATCCAGCCCCGTGTCTGGGGTGAGCATA
GGTGGGGAGGGCCCCAAGCTCACGTGAGCACGTTCTGGAAGTCTGACCCT
TAGGGAAAGAGGGAGTCAAGCCCATGGCCACTGGGATCACTCACAGGTGT
AACTCTCCACCTCAAAACCCTTCCAACTCCCAGAGCCTGTGCCTCTGGAG
GAGGTCCAATCGGTGGTGGAGCCAGAAGGTGGAGCAGTAGCTCCTGGTGG
AACCGTAACCCTGACCTGTGAAGTCCCTGCCCAGCCCTCTCCTCAAATCC
ACTGGATGAAGGATGGTGTGCCCTTGCCCCTTCCCCCCAGCCCTGTGCTG
ATCCTCCCTGAGATAGGGCCTCAGGACCAGGGAACCTACAGCTGTGTGGC
CACCCATTCCAGCCACGGGCCCCAGGAAAGCCGTGCTGTCAGCATCAGCA
TCATCGAACCAGGCGAGGAGGGGCCAACTGCAGGTGAGGGGTTTGATAAA
GTCAGGGAAGCAGAAGATAGCCCCCAACACATGTGACTGGGGGGATGGTC
AACAAGAAAGGAATGGAAGGCCCCAGAAAACCAGGAGGAAGAGGAGGAGC
GTG
sRAGE2 (511 bp)

(SEQ ID NO. 5)

GATCCCCGTCCCACCTTCTCCTGTAGCTTCAGCCCAGGCCTTCCCCGACA
CCGGGCCTTGCGCACAGCCCCCATCCAGCCCCGTGTCTGGGAACCTGTGC
CTCTGGAGGAGGTCCAATTGGTGGTGGAGCCAGAAGGTGGAGCAGTAGCT
CCTGGTGGAACCGTAACCCTGACCTGTGAAGTCCCTGCCCAGCCCTCTCC
TCAAATCCACTGGATGAAGGATGGTGTGCCCTTGCCCCTTCCCCCCAGCC
CTGTGCTGATCCTCCCCGAGATAGGGCCTCAGGACCAGGGAACCTACAGC
TGTGTGGCCACCCATTCCAGCCACGGGCCCCAGGAAAGCCGTGCTGTCAG
CATCAGCATCATCGAACCAGGCGAGGAGGGGCCAACTGCAGGTGAGGGGT
TTGATAAAGTCAGGGAAGCAGAAGATAGCCCCCAACACATGTGACTGGGG
GGATGGTCAACAAGAAAGGAATGGAAGGCCCCAGAAAACCAGGAGGAAGA
GGAGGAGGCGTG
sRAGE3 (698 bp)

(SEQ ID NO. 6)

GATCCCCGTCCCACCTTCTCCTGTAGCTTCAGCCCAGGCCTTCCCCGACA
CCGGGCCTTGCGCACAGCCCCCATCCAGCCCCGTGTCTGGGGTGAGCATA
GGTGGGGAGGGCCCCAAGCTCACGTGAGCACGTTCTGGAAGTCTGACCCT
TAGGGAAAGAGGGAGTCAAGCCCATGGCCACTGGGATCACTCACAAGTGT
AACTCTCCACCTCAAAACCCTTCCAACTCCCAGAGCCTGTGCCTCTGGAG
GAGGTCCAATTGGTGGTGGAGCCAGAAGGTGGAGCAGTAGCTCCTGGTGG
AACCGTAACCCTGACCTGTGAAGTCCCTGCCCAGCCCTCTCCTCAAATCC
ACTGGATGAAGGATGGTGTGCCCTTGCCCCTTCCCCCCAGCCCTGTGCTG
ATCCTCCCTGAGATAGGGCCTCAGGACCAGGGAACCTACAGCTGTGTGGC
CACCCATTCCAGCCACGGGCCCCAGGAAAGCCGTGCTGTCAGCATCAGCA
TCATCGAACCAGGCGAgGAGGGGCCAACTGCAGGCTCTGTGGGAGGATCA
GGGCTGGGAACTCTAGCCCTGGCCCTGGGGATCCTGGGAGGCCTGGGGAC
AGCCGCCCTGCTCATTGGGGTCATCTTGTGGCAAAGGCGGCAACGCCGAG
GAGAGGAGAGGAAGGCCCCAGAAAACCAGGAGGAAGAGGAGGAGCGTG

The characteristic features of the invention disclosed in the above description, the claims and the drawings may be essential, both individually and in any desired combination, for realising the invention in its various embodiments.

1-19. (canceled)

20. Use of HMGB and/or a nucleic acid that codes therefor and/or an interaction partner of HMGB that is preferably a natural interaction partner, and/or a nucleic acid that codes therefor as target molecule for the development and/or manufacture of a medicament for the treatment and/or prevention of endometriosis and/or for the development and/or production of a diagnostic agent for diagnosing endometriosis.

21. Use of HMGB, a nucleic acid that codes therefor or an interaction partner that is preferably a natural interaction partner, and/or a nucleic acid that codes therefor as target molecule for the development and/or the manufacture of a medicament, the medicament being a contraceptive.

22. The use according to claim 20 or 21 characterised in that the medicament comprises an agent which is selected from the group which comprises antibodies, peptides, anticalins, small molecules, antisense molecules, aptamers, Spiegelmers and RNAi molecules.

23. The use according to claim 22 characterised in that the agent enters into interaction with HMGB or with an interaction partner of HMGB that is preferably a natural interaction partner.

24. The use according to claim 22 characterised in that the agent enters into interaction with a nucleic acid that codes for HMGB and/or with a nucleic acid that codes for an interaction partner of HMGB that is preferably a natural interaction partner, in particular with mRNA, genomic nucleic acid or cDNA for HMGB.

25. Use of a polypeptide that enters into interaction with HMGB or with an interaction partner of HMGB that is preferably a natural interaction partner, for the manufacture or development of a medicament, the medicament being one that is selected from the group which comprises medicaments for the treatment and/or prevention of endometriosis and contraceptives, and/or for the manufacture or development of a diagnostic agent for diagnosing endometriosis.

26. The use according to claim 25 characterised in that the polypeptide is selected from the group which comprises antibodies against HMGB and HMGB binding polypeptides.

27. Use of a nucleic acid that enters into interaction with HMGB or with an interaction partner of HMGB that is preferably a natural interaction partner, for the manufacture or development of a medicament, the medicament being one that is selected from the group which comprises medicaments for the treatment and/or prevention of endometriosis and contraceptives, and/or for the manufacture or development of a diagnostic agent for diagnosing endometriosis.

28. The use according to claim 27 characterised in that the nucleic acid is selected from the group comprising aptamers and Spiegelmers.

29. Use of a nucleic acid that enters into interaction with a nucleic acid that codes for HMGB or for an interaction partner of HMGB that is preferably a natural interaction partner, for the manufacture or development of a medicament, the medicament being one that is selected from the group which comprises medicaments for the treatment and/or prevention of endometriosis and contraceptives, and/or for the production or development of a diagnostic agent for diagnosing endometriosis.

30. The use according to claim 29 characterised in that the nucleic acid entering into interaction is an antisense oligonucleotide, a ribozyme or RNAi.

31. The use according to claim 29 or 30 characterised in that the nucleic acid that codes for HMGB or for an interaction partner of HMGB that is preferably a natural interaction partner, is the respective cDNA or mRNA.

32. The use according to one of claim 20, 21, 25, 27 or 29 characterised in that HMGB is selected from the group which comprises HMGB1, HMGB2, HMGB3 and SP100-HMG.

33. The use according to one of claim 20, 21, 25, 27 or 29 characterised in that the interaction partner of HMGB, in particular HMGB1, is RAGE.

34. Use of RAGE or a derivative thereof for the development and/or manufacture of a medicament for the treatment and/or prevention of endometriosis and/or for the development and/or manufacture of a diagnostic agent for diagnosing endometriosis.

35. The use according to claim 34 characterised in that the RAGE derivative is an sRAGE or a translation product of a nucleic acid according to SEQ ID NO 4, 5 or 6.

36. A pharmaceutical composition comprising at least one agent which is selected from the group which comprises polypeptides that enter into interaction with HMGB, with a nucleic acid that codes therefor, with an interaction partner of HMGB that is preferably a natural interaction partner, and/or with a nucleic acid that codes therefor, nucleic acids that enter into interaction with HMGB or an interaction partner of HMGB that is preferably a natural interaction partner and nucleic acids that enter into interaction with nucleic acid(s) that codes or code for HMGB or for an interaction partner of HMGB that is preferably a natural interaction partner, and at least one pharmaceutically acceptable carrier, for the treatment and/or prevention of endometriosis and/or for contraception

37. A kit for the characterisation of the endometrial condition, in particular for the determination of the presence of a pregnancy or cycle interferences, comprising a polypeptide that enters into interaction with HMGB, with a nucleic acid that codes therefor, with an interaction partner of HMGB that is preferably a natural interaction partner, or with a nucleic acid that codes therefor, a nucleic acid that enter into interaction with HMGB, with a nucleic acid that codes therefor, with an interaction partner of HMGB that is preferably a natural interaction partner, or with a nucleic acid that codes therefor and/or a nucleic acid that enters into interaction with a nucleic acid that codes for HMGB and/or for an interaction partner of HMGB that is preferably a natural interaction partner.


SEQUENCE LISTING (11 sequences)

SEQ ID NO: 1 - DNA, 1207 bp, Homo sapiens
  misc_feature (1)..(1207): cDNA sequence of HMGB1
gagacagcgccggggcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagtttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaagttgcagctgttttgttgacattctgaatgcttctaagtaaatacaattttttttattaaaaaaaaaaaaaaaaaaaaaaaaa

SEQ ID NO: 2 - protein, 215 aa, Homo sapiens
  misc_feature (1)..(1207): cDNA sequence of HMGB1
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 3 - protein, 215 aa, Homo sapiens; identical to SEQ ID NO: 2
  MISC_FEATURE (1)..(215): Amino acid sequence of the HMGB1 protein (accession-no. S02826)

SEQ ID NO: 4 - DNA, 653 bp, Homo sapiens
  misc_feature: additional transcript RAGE1 (cDNA)
gatccccgtcccaccttctcctgtagcttcagcccaggccttccccgacaccgggccttgcgcacagcccccatccagccccgtgtctggggtgagcataggtggggagggccccaagctcacgtgagcacgttctggaagtctgacccttagggaaagagggagtcaagcccatggccactgggatcactcacaggtgtaactctccacctcaaaacccttccaactcccagagcctgtgcctctggaggaggtccaatcggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggtgaggggtttgataaagtcagggaagcagaagatagcccccaacacatgtgactggggggatggtcaacaagaaaggaatggaaggccccagaaaaccaggaggaagaggaggagcgtg

SEQ ID NO: 5 - DNA, 511 bp, Homo sapiens
  misc_feature: additional transcript RAGE2 (cDNA)
gatccccgtcccaccttctcctgtagcttcagcccaggccttccccgacaccgggccttgcgcacagcccccatccagccccgtgtctgggaacctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccccgagatagggcctcaggaccagggaacctacagctgtgtggccacccattccagccacgggccccaggaaagccgtgctgtcagcatcagcatcatcgaaccaggcgaggaggggccaactgcaggtgaggggtttgataaagtcagggaagcagaagatagcccccaacacatgtgactggggggatggtcaacaagaaaggaatggaaggccccagaaaaccaggaggaagaggaggagcgtg

SEQ ID NO: 6 - DNA, 698 bp, Homo sapiens
  misc_feature: additional transcript RAGE3 (cDNA)
gatccccgtcccaccttctcctgtagcttcagcccaggccttccccgacaccgggccttgcgcacagcccccatccagccccgtgtctggggtgagcataggtggggagggccccaagctcacgtgagcacgttctggaagtctgacccttagggaaagagggagtcaagcccatggccactgggatcactcacaagtgtaactctccacctcaaaacccttccaactcccagagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtg

SEQ ID NO: 7 - DNA, 23 bp, Artificial Sequence
  Sequencing primer (laboratory code M13uni)
cccagtcacgacgttgtaaaacg

SEQ ID NO: 8 - DNA, 23 bp, Artificial Sequence
  Sequencing primer (laboratory code M13rev)
agcggataacaatttcacacagg

SEQ ID NO: 9 - DNA, 34 bp, Artificial Sequence
  Adaption primer (laboratory code AP2)
aaggatccgtcgacatcttttttttttttttttt

SEQ ID NO: 10 - DNA, 27 bp, Artificial Sequence
  Detection primer (laboratory code PrimerRAGE2up)
gatccccgtcccaccttctcctgtagc

SEQ ID NO: 11 - DNA, 30 bp, Artificial Sequence
  Detection primer (laboratory code RAGE2lo)
cacgctcctcctcttcctcctggttttctg